(19) United States
(12) Patent Application Publication
(10) Pub. No.: US 2018/0099018 A1
(43) Pub. Date: Apr. 12, 2018

(54) MAHONIA BEALEI EXTRACT WITH ANTIBACTERIAL INFLAMMATION-DIMINISHING AND GRANULATION-PROMOTING FUNCTIONS AS WELL AS PREPARATION METHOD AND APPLICATION THEREOF

(71) Applicant: Guangdong Hospital of Traditional Chinese Medicine, Guangzhou (CN)

(72) Inventors: Yi LIN, Guangzhou (CN); Zhiyu WANG, Guangzhou (CN); Honglin SITU, Guangzhou (CN); Neng WANG, Guangzhou (CN)

(21) Appl. No.: 15/729,651

(22) Filed: Oct. 10, 2017

(30) Foreign Application Priority Data
Oct. 10, 2016 (CN) 201610883949.X

(51) Int. Cl. A61K 36/29

(52) U.S. Cl. CPC A61K 36/29; A61K 2236/51; A61K 2236/53; A61K 2236/333

(57) ABSTRACT

The present invention discloses an application of a mahonia bealei extract alone serving as active ingredient in preparation of drugs for treating chronic inflammatory traumas. The chronic inflammatory traumas comprise traumas initiated by chronic mastitis, diabetic foot, burn, chronic ulcer or Crohn's disease. A preparation method of the mahonia bealei extract comprises steps: taking stems and leaves of a plant mahonia bealei as raw materials, extracting with water, ethanol or a water-containing alcohol, filtering, concentrating to a small volume, and performing freeze drying for later use. The present invention has beneficial effects as follows: a drug preparation process is simple and reasonable in operation; an application value of the mahonia bealei extract in treatment of a chronic inflammatory wound is clarified; and medicinal and drug efficacy study is developed, effective protection is realized on a utilization basis, and effects of off-site conservation and sustainable development can be achieved.

### TECHNICAL FIELD

The present invention relates to the technical field of traditional Chinese medicines, and particularly relates to a preparation method for extracting an effective part from a natural plant mahonia bealei (wintergreen barberry root) and an application of the mahonia bealei in chronic inflammatory traumas.

### BACKGROUND

On a global scale, probably about more than two million patients suffer from chronic inflammatory wounds, and this quantity is progressively increasing at a speed of 10-15% per year. Chronic impaired healing wounds are common frequently-occurring diseases which severely endanger human health, such as diabetic foot ulcer, Crohn's disease, irritable bowel syndrome, granulomatous lobular mastitis, etc. Since underlying diseases are complicated, with disease sites accompanying with lots of inflammatory cells and factors being infiltrated, severe tissue damage and even necrosis are caused and at the same time curative effects of antibiotic or hormone therapy are limited. How to accelerate healing of chronic inflammatory wounds becomes a clinical problem which urgently needs to be solved in recent years.

Traditional Chinese medicine is a way to solve refractory diseases in the world, and immunoregulation of a body of a patient suffering from chronic inflammations is a unique advantage of the traditional Chinese medicine. In recent ten years, lots of experiments show that multiple compounds and single traditional Chinese medicines can balance immunologic functions of patients from aspects of inhibiting an NF-kB signal pathway, blocking burst of reactive oxygen free radicals, balancing disordered cell factors, inhibiting MAPK growth signal pathways and the like, and have multi-link, multi-target and overall regulation characteristics. For example, the above traditional Chinese medicines may include dandelion, lonicera japonica, houttuynia cordata, etc. However, single traditional Chinese medicines with antibacterial, inflammation-diminishing and granulation-promoting functions are not common.

Mahonia bealei is also known as wintergreen barberry root and is bitter in taste, cold in nature and non-toxic, and a medicinal part is mainly a leaf of the mahonia bealei. At present, study on pharmacological activity of the mahonia bealei is rare, and no reports on its anti-inflammatory activity and sterilization activity exist. Chemical component analysis shows that the plant takes alkaloid chemical components as major components. At present, 25 alkaloids have been separated and identified. In addition to three main alkaloids, that is, berberine, palmatine and jateorhizine, the alkaloids further include the following chemical components: tetrahydrojatrorrhizine, columbamine, oxyberberine, berbamine, coptisine, oxyacanthine, baluchistine, obamegine, aromoline, aquifoline, magnoflorine, magnolamine, obaberine, isocorydine, corydine, corytuberine, fortune meadowrue herb, thalrugosine, glaucine, isoboldine, isothebaine and isotetrandrine. However, active medicinal composition and medicine efficacy of the wintergreen barberry root have never been reported at present. On this basis, the invention performs comprehensive study on chemical composition, effective parts and biological effects of the mahonia bealei.

### SUMMARY

One purpose of the present invention is to provide a preparation method of a mahonia bealei extract.

Another purpose of the present invention is to provide an application of mahonia bealei in drugs for promoting healing of chronic inflammatory wounds.

The following technical solutions are specifically adopted in the present invention: An application of the mahonia bealei extract in preparation of drugs, the mahonia bealei extract alone serves as an active ingredient in preparation of drugs for treating chronic inflammatory traumas.

The chronic inflammatory traumas comprise traumas initiated by any one of chronic mastitis, diabetic foot, burn, chronic ulcer and Crohn's disease.

The mahonia bealei extract are prepared into tablets, capsules, injections, external solutions, lotions, liniments, enemas, ointments, pastes, patches and sustained-release or controlled-release preparations with medically acceptable carriers or excipients.

A preparation method of the mahonia bealei extract comprises the steps: taking stems or leaves of a mahonia bealei plant as raw materials, extracting with water, ethanol or a water-containing alcohol, filtering, concentrating to a small volume, and performing freeze drying for later use.

The mahonia bealei extract is preferably extracted with water of 60-120° C. for 1-8 times, more preferably 2-5 times.

The mahonia bealei ethanol extract is preferably extracted with 10-95% of ethanol, more preferably extracted with 20-80% of ethanol, further preferably extracted with 40-70% of ethanol; extraction manners can be selected from percolation, reflux, impregnation or ultrasonic extraction; and preferably extraction is performed for 1-8 times, more preferably 2-5 times.

The mahonia bealei extract can be further preferably enabled to pass through macroporous resin, firstly elute with water, then elute with ethanol having gradually increased concentrations sequentially, and collect a high-concentration ethanol eluate, that is, an active site of mahonia bealei.

The mahonia bealei extract in the present invention is subjected to in-vitro anti-inflammatory experiment screening, an in-vitro anti-inflammatory model is established by utilizing lipoproteins (LPS) induced mice macrophage RAW264.7, production of inflammatory factors such as cyclooxygenase-2 (COX-2), nitric oxide synthase (iNOS), IL-1β and IL-6 is detected, and the lower the validation factor concentration is, the higher the activity is. Experimental results show that the aqueous extract and ethanol extract of the mahonia bealei in the present invention have effects on inhibiting expressions of the inflammatory factors. The mahonia bealei in the present invention is subjected to a molecular mechanism experiment. Each dosage group has different degrees of effects on inhibiting activation of NF-kB pathways, and transport of the NF-kB into a cell nucleus can be obviously blocked, thereby inhibiting expressions of downstream inflammatory factors.

An antibacterial experiment of the mahonia bealei extract in the present invention shows that each dosage group of the mahonia bealei extract has good bacteriostatic activity on staphylococcus aureus, enterococcus faecalis and Escherichia coli and does not have any inhibitory effect on pseudomonas aeruginosa, klebsiella pneumonia and enterobacter cloacae.

An angiogenesis experiment of the mahonia bealei extract in the present invention shows that each dosage group of the mahonia bealei extract can achieve effects of obviously promoting proliferative activities of human vascular endothelial cells, promoting migration and angiogenesis capabilities and promoting fibroblasts to secrete type I and III collagens. A chicken aortic ring experiment shows that the mahonia bealei extract has angiogenesis promoting ability in vivo.

An in-vivo clinical individual experiment of the mahonia bealei extract in the present invention shows that the mahonia bealei extract can achieve effects on inhibiting bacterial growth on a mastitis wound surface, eliminating inflammations and relieving red and swollen heat pain phenomena caused by the inflammations. Any discomfort of the patient is avoided, and effects of promoting growth of newborn granulation tissues and promoting healing of inflammatory wounds can be achieved.

In conclusion, compared with the prior art, the present invention has beneficial effects as follows:

(1) a drug preparation process is simple and reasonable in operation;

(2) through in-vivo/vitro anti-inflammatory pharmacological experiments, an application value of the mahonia bealei extract in treatment of the chronic inflammatory wound is clarified, and a theoretical and experimental basis is provided for clinical development of the mahonia bealei extract; and

(3) study on economic value of the mahonia bealei is still almost in blank, medicinal and drug efficacy study is developed, effective protection is realized on a utilization basis, and effects of off-site conservation and sustainable development can be achieved. The mahonia bealei is wide in distribution and strong in adaptability. Once the mahonia bealei is utilized, large-scale cultivation can be realized, plant resources are greatly enriched, and economic income of peasants can be increased.

### BRIEF DESCRIPTION OF DRAWINGS

FIG. 1 shows an in-vitro anti-inflammatory activity experiment of a mahonia bealei extract.

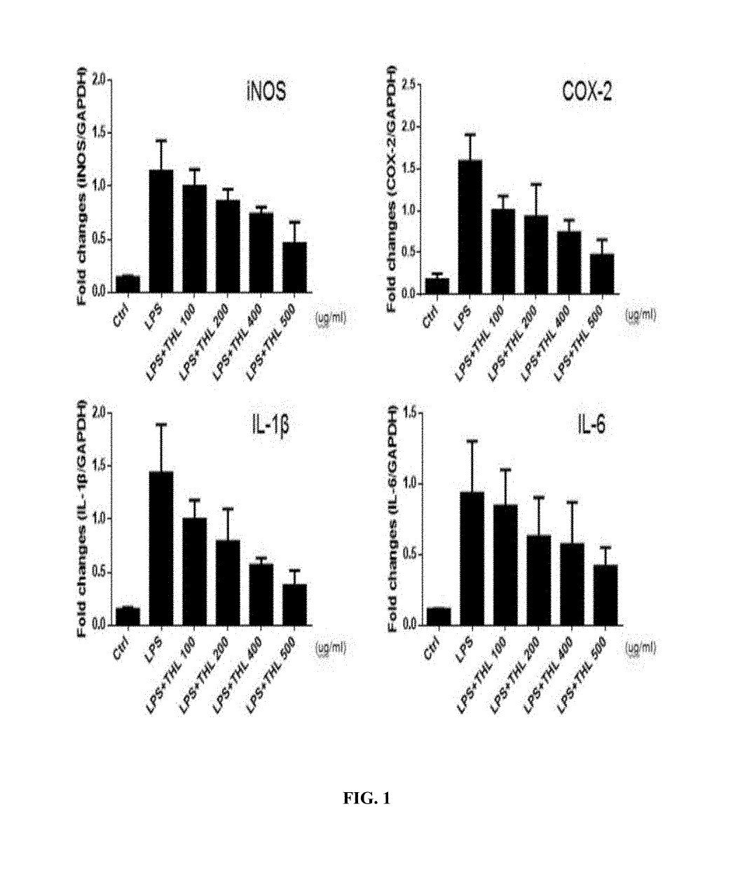

FIG. 2 shows an in-vivo/vitro angiogenesis-promoting experiment of a mahonia bealei extract.

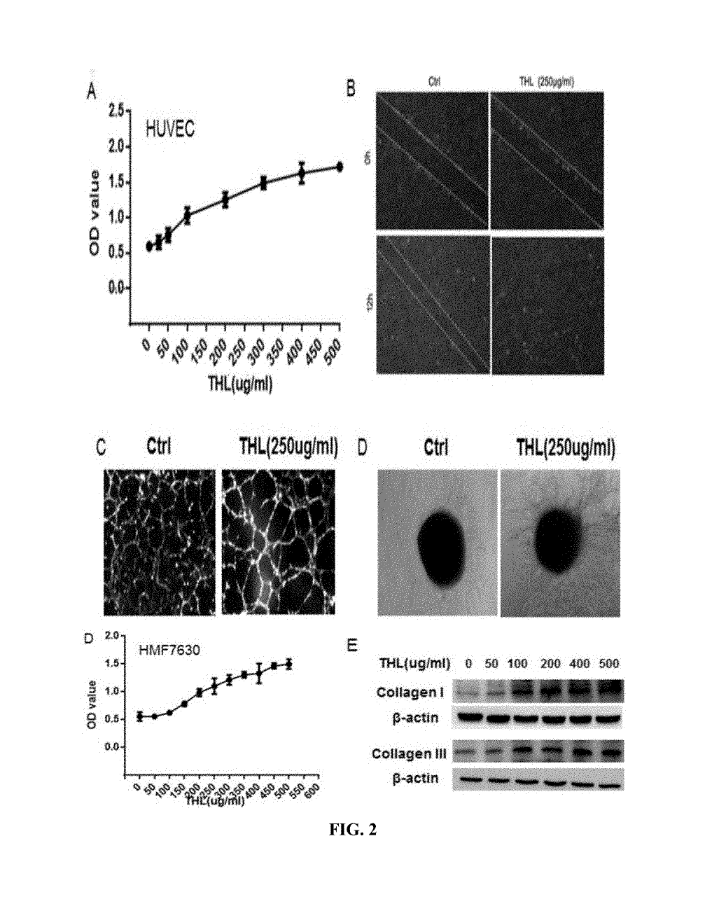

### DETAILED DESCRIPTION

The present invention is further described in detail in combination with specific embodiments, but contents of the present invention are not limited to illustrated embodiments only.

### Embodiment 1 Preparation of Mahonia Bealei

The preparation comprises the following steps:

taking 100 g of stem leaves of mahonia bealei, decocting and extracting with 10 times that of water (W/V) twice at a frequency of one hour each time, merging extracting solutions of two times, performing reduced pressure concentration, and performing freeze drying to obtain 12.3 g of crude extract; or taking 100 g of stem leaves of mahonia bealei, refluxing and extracting with 60% (W/V) of ethanol in an amount of 10 times twice at a frequency of one hour each time, merging the extracting solutions of two times, performing reduced pressure concentration, and performing freeze drying to obtain 13.7 g of crude extract.

The mahonia bealei extract is subjected to component analysis by adopting an HPLC technology. A separation column is UltimateAQ-C18 (250 mm×4.6 mm, 5 μm), and a separation solution is composed of acetonitrile (A) and water (B). A separation solution is 5%-8% Aat0-5 min, 8%-14% Aat5-30 min, 14%-50% Aat30-55 min, 50%-90% Aat55-60 min, and chemical fingerprint chromatography is observed at 250 nm. Berberine serves as a standard substance, and a concentration of the berberine is 0.014-0.018 mg/ml in the mahonia bealei. Meanwhile, chemical properties of three main peaks are identified by adopting an LC-MS technology, and the three main peaks are measured to be respectively chlorogenic acid, jateorhizine and magnoflorine.

### Embodiment 2 Cytotoxicity Test of Mahonia Bealei Extract

An MTT method is a method for detecting cell survival and growth. A detection principle is as follows: succinodehydrogenase in living cell mitochondria can enable exogenous MMTT to be reduced into water-insoluble bluish violet crystalline formazan and deposited in cells, while dead cells do not have the above function. The formazan in the cells can be dissolved by dimethyl sulfoxide (DMSO), a light absorption value of the formazan is measured at 540-720 nm by using an enzyme linked immunosorbent assay instrument, and the quantity of the living cells can be indirectly reflected. An amount of the formed MTT crystals is in direct proportion to cell population in a certain cell population range. The method has been widely applied to activity detection of some bioactive factors, large-scale antitumor drug screening, cytotoxicity test, tumor radiosensitivity measurement and the like. The method has characteristics of high sensitivity and economical efficiency. However, macrophage is an important inflammatory factor secreting cell, and its cell function is closely related to inflammations.

Experimental samples are aqueous extracts and 60% of ethanol extracts of the mahonia bealei, and positive control drugs are lipoproteins (LPS) and extract and LPS are sequentially added according to a concentration gradient.

The method comprises the steps as follows:

inoculating mice macrophage Raw264.7 with a good logarithmic phase state in a 96-well plate, adding the tested drug, and respectively culturing for 48 hours; adding 5 mg/ml MTT into the 96-well plate according to an amount of 10 μl per well, reacting in an incubator for 4 hours, absorbing a supernatant, adding the DMSO according to an amount of 100 μl per well, shaking out for 5 min, measuring the light absorption value of each well by using the enzyme linked immunosorbent assay instrument, measuring a wavelength to be 570 nm, and calculating a down-inhibition rate.

Experimental results show that the positive control drugs LPS can obviously promote cell proliferation of the Raw264.7, while the aqueous extracts and ethanol extracts of the mahonia bealei do not have any obvious inhibitory effect in a concentration range of 1-500 ug/ml, thereby proving that the mahonia bealei extract does not have any obvious cytotoxicity.

### Embodiment 3 In-Vitro Anti-Inflammatory Activity Experiment of Mahonia Bealei Extract

COX-2, iNOS, IL-1β and IL-6 are extremely important inflammatory factor products and achieve important effects in aspects of regulating pain, red and swollen of inflammatory wounds and the like. The higher the expression content is, the more serious the inflammatory response is. On the contrary, if an expression level is decreased, a degree of the inflammatory response is correspondingly decreased. Therefore, the expression level is an important indicator for measuring the degree of the inflammatory response. An in-vitro anti-inflammatory model is established by utilizing the LPS-induced mice macrophage RAW264.7, and changes of four indexes after addition of the mahonia bealei extract are detected by a Real-time PCR method. Experimental results show that expression contents of the four factors present a decrease in dose-dependent after the aqueous extracts and ethanol extracts of the mahonia bealei are added, indicating that the mahonia bealei extract has the anti-inflammatory activity.

### Embodiment 4 In-Vitro Antibacterial Activity Experiment of Mahonia Bealei Extract

The method comprises the steps as follows:

taking Gram staining positive bacteria such as staphylococcus aureus and enterococcus faecalis and Gram staining negative bacteria such as pseudomonas aeruginosa, Escherichia coli, klebsiella species and enterobacter cloacae as study objects, and measuring growth inhibitory activity of each concentration of the mahonia bealei extract on the bacteria by adopting an LB medium agar block method; preparing suspension from a tested strain, uniformly coating the suspension onto an LB culture plate, and inverting the culture plate in an incubator of 37° C. for incubating; and punching sterile filter paper into circular paper scraps with a diameter of 0.5 cm by using a sterile puncher after drying the LB culture plate, then immersing the paper scraps into a mahonia bealei extracting solution of each concentration, subsequently putting the circular paper scraps into an LB bacterium culture plate according to the concentration gradient, culturing overnight at 37° C., measuring a diameter of an inhibition zone on the next day, and evaluating the antibacterial activity of the mahonia bealei.

Experimental results show that each dosage group of the mahonia bealei extract has good antibacterial activity on the staphylococcus aureus, the enterococcus faecalis and the Escherichia coli and present dose-dependent, but does not have any inhibitory effect on the pseudomonas aeruginosa, the klebsiella pneumonia and the enterobacter cloacae.

### Embodiment 5 In-Vivo/Vitro Angiogenesis-Promoting Experiment of Mahonia Bealei Extract

Angiogenesis is a necessary condition for forming granulation. The method in the present invention comprises the steps as follows: taking human umbilical vein vascular endothelial cells (HUVEC) as a study object, studying whether the mahonia bealei extracting solution can promote proliferation of the HUVEC by adopting the MTT method, and evaluating a migration promoting effect of the mahonia bealei extract on the HUVEC by adopting a scratch test; inducing the HUVEC to form a tube type by utilizing Matrigel on this basis, and evaluating whether the mahonia bealei extract can accelerate the angiogenesis process; and finally, determining an effect of the mahonia bealei extract for promoting chick embryo angiogenesis by adopting a chicken aortic ring experiment; dissecting the chick embryo, taking an aorta of the chick embryo, cutting into a ring, placing in a 12-well plate laid with Matrigel, then adding the mahonia bealei extracts of different concentrations, and observing a proliferation rate and a growth length of vascular endothelial cells around the aorta.

Experimental results show that each dosage group of the mahonia bealei extract can achieve effects of promoting proliferative activities of human vascular endothelial cells and promoting migration and angiogenesis capabilities, and the chicken aortic ring experiment shows that the mahonia bealei extract can promote in-vivo angiogenesis. On the other hand, human mammary fibroblasts HMF7630 are treated by using the mahonia bealei extract, discovering that the mahonia bealei extract can obviously promote proliferation of the HMF7630 and meanwhile, secretion of type I and III collagens in cells is increased, proving that the mahonia bealei extract can promote healing of wounds and increase a growth speed of granulation tissues.

Embodiment 6 Molecular mechanism experiment of mahonia bealei extract

NF-kB pathway activation is a main activation pathway in an inflammatory response process and controls expressions of the inflammatory factors of COX-2, iNOS, IL-1β and IL-6 in an upstream level. In the present invention, influences of the mahonia bealei extract on the NF-kB pathway are detected by adopting a western blotting technology and a laser confocal technology.

Experimental results show that the mahonia bealei extract can inhibit activation of the NF-kB in a dose-dependent manner, inhibits an extent of phosphorylation and has inhibitory effects on phosphorylation of upstream molecules IKKα/β and IKBα. A laser confocal experiment shows that the mahonia bealei extract can inhibit transport of the NF-kB into a cell nucleus, thereby blocking the NF-kB from activating transcription of downstream genes.

### Embodiment 7 Clinical Effect Experiment of Mahonia Bealei

138 patients suffering from chronic granulomatous lobular mastitis are incorporated, with 71 patients being treated by traditional Chinese medicine taking the mahonia bealei extract as a principle component, 67 patients being treated by western medicine surgeries, all the patients being female, an average age of the two groups being 33.29±7.47, the minimum age being 16, the maximum age being 69; age composition, clinical symptoms, courses of diseases, clinical stages and other aspects of the two groups do not have any statistic difference, and a comparison base is consistent. Surgical treatment is composed of segmental mastectomy, extended lumpectomy and simple mastectomy. The traditional Chinese medicine treatment group takes cleaning, drainage and wet dressing with the mahonia bealei solution as a principle manner after an inflammatory debridement.

(1) criteria of curative effects:

cured:

improved:

uncured:

(2) criteria of breast shape changes:

excellent:

good:

fair:

poor:

See treatment effects in Table 1 and Table 2.

TABLE 1
Clinical curative effects of mahonia bealei

| | | Curative effects | | |
|---|---|---|---|---|
| | Number | Cured | Improved | Uncured |
| Groups | of cases | (%) | (%) | (%) |
| Traditional Chinese | 71 | 66 (93%) | 5 (7%) | 0 (%) |
| medicine treatment | | | | |
| group | | | | |
| Surgical treatment | 67 | 62 (92.5%) | 5 (7.5%) | 0 (%) |
| group | | | | |

TABLE 2
Breast shape changes in treatment group through mahonia bealei

| | | Curative effects | | | |
|---|---|---|---|---|---|
| | Number | Excellent | Good | Fair | Poor |
| Groups | of cases | (%) | (%) | (%) | (%) |
| Traditional | 71 | 65 (91.5%) | 5 (7.0%) | 1 (1.4%) | 0 (0) |
| Chinese | | | | | |
| medicine | | | | | |
| treatment group | | | | | |
| Surgical | 67 | 4 (6.0) | 18 (26.9) | 36 (53.7) | 9 (13.4) |
| treatment group | | | | | |

It is found through statistics that the two groups do not have obvious difference on the curative effects of treatment, but the breast shape evaluation in the traditional Chinese medicine treatment group is excellent and is obviously better than that in the surgical treatment group (P<0.05). Moreover, no obvious toxic, side reaction or adverse event are found in the treatment process, and no skin allergy phenomenon does exist.

What is claimed is:

1. An application of a mahonia bealei extract in preparation of drugs; wherein the mahonia bealei extract alone serves as an active ingredient in preparation of drugs for treating chronic inflammatory traumas.

2. The application of the mahonia bealei extract in preparation of drugs according to claim 1, wherein the chronic inflammatory traumas comprise traumas initiated by any one of chronic mastitis, diabetic foot, burn, chronic ulcer and Crohn's disease.

3. The application of the mahonia bealei extract in preparation of drugs according to claim 1, wherein the mahonia bealei extract are prepared into tablets, capsules, injections, external solutions, lotions, liniments, enemas, ointments, pastes, patches and sustained-release or controlled-release preparations with medically acceptable carriers or excipients.

4. A preparation method for preparing the mahonia bealei extract according to claim 1, comprising the following steps:
step 1, taking stems or leaves of a mahonia bealei plant as raw materials;
step 2, extracting with water, ethanol or a water-containing alcohol;
step 3, filtering;
step 4, concentrating to a small volume; and
step 5, performing freeze drying for later use.

5. The preparation method for preparing the mahonia bealei extract according to claim 4, wherein extraction is performed with water of 60-120° C. for 1-8 times in the step 2.

6. The preparation method for preparing the mahonia bealei extract according to claim 5, wherein the extraction is performed with water of 60-120° C. for 2-5 times in the step 2.

7. The preparation method for preparing the mahonia bealei extract according to claim 4, wherein extraction is performed with ethanol having a volume concentration of 10-95% for 1-8 times in the step 2; extraction manners comprise percolation, reflux, impregnation or ultrasonic extraction.

8. The preparation method for preparing the mahonia bealei extract according to claim 7, wherein the extraction is performed with ethanol having a volume concentration of 40-70% for 2-5 times in the step 2; extraction manners comprise percolation, reflux, impregnation or ultrasonic extraction.

9. The preparation method for preparing the mahonia bealei extract according to claim 4, wherein the step 5 comprises the following sub-steps: enabling the mahonia bealei extract to pass through macroporous resin, firstly eluting with water, then eluting with ethanol having gradually increased concentrations sequentially, collecting a high-concentration ethanol eluate, that is, an active site of mahonia bealei.
